(19) United States
(12) Patent Application Publication
(10) Pub. No.: US 2017/0044625 A1
(43) Pub. Date: Feb. 16, 2017

(54) METHODS OF DIAGNOSING AND TREATING aCML and CNL

(71) Applicant: OREGON HEALTH & SCIENCE UNIVERSITY, Portland, OR (US)

(72) Inventors: Jeffrey Tyner, Portland, OR (US); Julia Maxson, Portland, OR (US); Brian Druker, Portland, OR (US)

(21) Appl. No.: 15/336,570

(22) Filed: Oct. 27, 2016

Related U.S. Application Data
(63) Continuation of application No. 14/245,106, filed on Apr. 4, 2014.
(60) Provisional application No. 61/809,055, filed on Apr. 5, 2013.

(51) Int. Cl. C12Q 1/68; A61K 31/506; A61K 31/519

(52) U.S. Cl. CPC C12Q 1/6886; A61K 31/519; A61K 31/506; C12Q 2600/106; C12Q 2600/156

(57) ABSTRACT

Disclosed herein are methods and kits used in treating a cancer characterized by aberrant activity of CSF3R. These methods involve detecting a mutation in exon 14 of CSF3R such as a mutation of T615 or T618 and treating the subject with a JAK inhibitor and/or detecting a mutation in exon 17 of CSF3R and treating with dasatinib or tyrosine kinase inhibitor with one or more targets in common with dasatinib.

### PRIORITY CLAIM

This application claims the benefit of U.S. Provisional Patent Application No. 61/809,055 filed 5 Apr. 2013; incorporated by reference herein.

### ACKNOWLEDGEMENT OF GOVERNMENT SUPPORT

This invention was made with the support of the United States Government under the terms of Grant Numbers 1 RC1 CA146107-01 and T-32 HL07781 awarded by the National Institutes of Health. The United States Government has certain rights in this invention.

### FIELD

Generally, the field is methods of treating subjects with cancer. More specifically, the field is methods of treating subjects with cancers characterized by aberrant tyrosine kinase activity.

### BACKGROUND

Small-molecule kinase inhibitor therapy has improved patient outcomes for certain types of cancer with kinase pathway dependence caused by defined genetic abnormalities (Druker B J et al, N Engl J Med 355, 2408-2417 (2006); and Krause D S and Van Etten R A N Engl J Med 353, 172-187 (2005), both of which are incorporated by reference herein.) Extrapolation of this paradigm to other malignancies requires knowledge of operationally important mutant genes that result in corresponding kinase pathway activation. Despite advances in our understanding of the molecular pathobiology of certain types of hematologic malignancies, many of these disorders are still diagnosed on the basis of neoplastic cell type and additional exclusionary criteria. Chronic neutrophilic leukemia (CNL) and atypical CML (aCML) are both rare hematologic neoplasms characterized by leukocytosis and hypercellular bone marrow comprised predominantly of granulocytic cells, absence of the Philadelphia chromosome (t(9;22); BCR-ABL1), and absence of platelet-derived growth factor receptor A/B (PDGFRA/B) or fibroblast growth factor receptor 1 (FGFR1) gene rearrangements.

CNL is diagnosed based on expansion of neutrophils in both the blood and bone marrow (segmented neutrophils and band forms >80% of white blood cells (WBC)) and is classified as a myeloproliferative neoplasm (MPN) according to World Health Organization (WHO) diagnostic criteria. Cases of aCML exhibit granulocytic dysplasia and increased numbers of neutrophil precursors in both the peripheral blood and the bone marrow (typically 10% of WBCs) and are therefore classified as one subtype of the WHO category of myelodysplastic/myeloproliferative neoplasms (MPN) (Bain B J et al, WHO Classification of Tumors of Haematopoietic and Lymphoid Tissues, 4^{th} ed, Lyon: IARC Press pp 38-39 (2008) and Vardiman J W et al, Ibid pp 80-81, both of which are incorporated by reference herein). Occasional cases of CNL (Froberg M K et al, Leukemia 12, 623-626 (1998) and Matano S et al, Am J Hematol 54, 72-75 (1997); both of which are incorporated by reference herein) and a majority of aCML cases are reported to exhibit non-specific cytogenetic abnormalities (Hernandez J M et al, Ann Oncol 11, 441-444 (2000); incorporated by reference herein) or infrequently the JAK2 V617F mutation (Baxter E J et al, Lancet 365, 1054-1061 (2005) and Steensma D P et al, Blood 106, 1207-1209 (2005); both of which are incorporated by reference herein), revealing the clonal nature of these diseases.

While certain MPN subtypes have either been operationally defined by molecular abnormalities (e.g. BCR-ABL1 in CML) or are characterized by a high frequency of specific genetic abnormalities (e.g. JAK2 V617F in polycythemia vera (PV), essential thrombocythemia (ET), and primary myelofibrosis (PMF) (Baxter E J et al, 2005 supra, James C et al, Nature 434, 1144-1148 (2005); Jones A V et al, Blood 106, 2162-2168 (2005); Kralovics R et al, N Engl J Med 352, 1779-1790 (2005) and Levine R L et al, Cancer Cell 7, 387-397 (2005); all of which are incorporated by reference herein); KIT D816V in systemic mastocytosis Longley B J et al, Nat Genet 12, 312-314 (1996) and Nagata H et al, Proc Natl Acad Sci USA 92, 10560-10564 (1995); both of which are incorporated by reference herein), the genetic basis of both CNL and aCML remains unknown.

CSF3R is the receptor for colony stimulating factor 3 (CSF3; GCSF), and is thought to play a prominent role in the growth and differentiation of granulocytes (Beekman R and Touw I P, Blood 115, 5131-5136 (2010) and Liu F et al, Immunity 5, 491-501 (1996); both of which are incorporated by reference herein). CSF3R mutations have been described in patients with severe congenital neutropenia, which can evolve into acute myeloid leukemia (AML) (Dong F et al, N Engl J Med 333, 487-493 (1995), Dong F et al, Proc Natl Acad Sci USA 91, 4480-4484 (1994) and Germeshausen M et al, Blood 109, 93-99 (2007); all of which are incorporated by reference herein). It was recently reported that a patient with congenital neutropenia developed a secondary CSF3R mutation at the time of AML transformation (Beekman R et al, Blood 119, 5071-5077 (2012), incorporated by reference herein). These previously described nonsense/frameshift mutations truncate the cytoplasmic tail of CSF3R, impair its internalization, and alter its interactions with proteins such as SHP-1/2 and SOCS family members (Dong F et al, J Immunol 167, 6447-6452 (2001); van de Geijn G J et al, Blood 104, 667-674 (2004); and Ward A C et al, Blood 93, 447-458 (1999); all of which are incorporated by reference herein). These structural and functional alterations are thought to perturb the capacity of CSF3R to regulate granulocyte differentiation and to increase granulocytic proliferative capacity (Hermans M H et al, Blood 92, 32-39 (1998); Hunter M G et al, Blood 95, 2132-2137 (2000); and Mitsui T et al, Blood 101, 2990-2995 (2003); all of which are incorporated by reference herein). CSF3R signals through the JAK/STAT pathway, the non-receptor tyrosine kinase SYK (Corey S J et al, J Biol Chem 273, 3230-3235 (1998) and Corey S J et al Proc Natl Acad Sci USA 91, 4683-4687 (1994); both of which are incorporated by reference herein), and the SRC family kinase LYN, which was recently shown to be mediated by the phosphatase SHP-2 and the adaptor protein GAB2 (Futami M et al, Blood 118, 1077-1086 (2011) and Zhu Q S et al, Blood 103, 3305-3312 (2004); both of which are incorporated by reference herein). With the exception of isolated case reports (Plo I et al, J Exp Med 206, 1701-1707 (2009); incorporated by reference herein) mutations in CSF3R have not been observed in cases of de novo leukemia. Clearly, new methods of diagnosing leukemia and determining a path of treatment, particularly for CNL and aCML are needed.

### SUMMARY

Disclosed herein are methods of diagnosing a subject as having aCML or CNL. Such methods include collecting a sample comprising leukocytes from the patient, purifying nucleic acids from those samples, and identifying a mutation in the nucleic acids encoding exon 14 and/or exon 17 of CSF3R (SEQ ID NO: 1 herein.) The mutation results in any one of the following amino acid mutations either alone or in combination: D771fs, G683R, S783fs, T615A, T618I, Y752X, E808K, and W791X. The presence of one or more of those mutations signifies that the patient has aCML or CNL. The methods may further comprise amplifying exon 14 and/or exon 17 by nucleic amplification such as polymerase chain reaction. The disclosed methods may also be used to signify that an aCML or CNL is sensitive to a tyrosine kinase inhibitor such as dasatinib or a JAK1 inhibitor such as ruxolitinib.

Also disclosed herein are methods of treating cancer in a subject, wherein the cancer is characterized by aberrant CSF3R activity. The method involves identifying a nucleic acid mutation in the sequence encoding exon 14 and/or exon 17 of CSF3R (SEQ ID NO: 1) that results in one or more of the following amino acid mutations, either alone or in combination: D771fs, G683R, S783fs, T615A, T618I, Y752X, E808K, and W791X. The method further involves administering an effective dose of dasatinib or a JAK1 inhibitor such as ruxolitinib.

Also disclosed herein are kits used in facilitating the disclosed methods.

### BRIEF DESCRIPTION OF THE DRAWINGS

FIG. 1A-1F collectively show the sensitivity to inhibition and/or silencing of kinases in CNL patient specimens with transforming mutations in CSF3R.

FIG. 1A is a graphic representation of the location and recurrence of CSF3R mutations found in human leukemia patient samples. The mutation locations and number of observations in this study are indicated by black circles. The Q741X mutation was found in an AML sample and one of the T618I mutations was found in an ETP-T-ALL.

All other mutations were observed in CNL/aCML specimens as described in Table 2. Two additional mutations (Q739X and T618I; not depicted in Figure) have been reported in AML specimens sequenced by The Cancer Genome Atlas. All CSF3R coordinates are numbered according to Ensembl conventions, which differ from historical CSF3R numbering due to inclusion of the 23 amino acid signal peptide in Ensembl.

FIG. 1B is a bar graph depicting the response of white blood cells from a patient with CNL (patient #3 from Tables 2 and 3; CSF3R S783fs) to a panel of 66 small molecule kinase inhibitors. The IC₅₀ of this patient specimen for each drug is plotted as a percentage of the median IC₅₀ for each drug from a cohort of 150 leukemia patient samples. A patient specimen is considered hypersensitive to inhibitors where the IC₅₀ is less than 20% of the median IC₅₀ for that inhibitor from the entire cohort. As such, this particular specimen was hypersensitive to dasatinib (green) and insensitive to JAK kinase inhibitors (red).

FIG. 1C is a bar graph depicting the response of white blood cells from CNL patient #3 to siRNAs directed against all known tyrosine kinases. Silencing of TNK2 and FGR resulted in a significant decrease in cell viability. Values represent mean percent cell viability±s.e.m. In addition to CSF3R S783fs, Patient #3 also exhibited a minority of clones with a CSF3R S783fs/T615A compound mutation, but this small percentage of cells would not impact the drug/siRNA sensitivity in short-term assays.

FIG. 1D is a bar graph depicting the response of white blood cells from a patient with ETP-T-ALL (CSF3R T618I) against a panel of 66 small molecule kinase inhibitors as in FIG. 1B. These cells were insensitive to dasatinib (green) and sensitive to JAK kinase inhibitors (red).

FIG. 1E is a line graph depicting the growth of Ba/F3 cells infected with murine retrovirus expressing WT CSF3R, membrane proximal mutations, or truncation mutations. Uninfected parental Ba/F3 cells and empty vector infected Ba/F3 cells were used as controls. IL-3 was removed from the Ba/F3 cells and viable cell numbers were counted over a ten-day period.

FIG. 1F is an image of an immunoblot of Ba/F3 cells expressing either T618I or S783fs CSF3R before or after IL-3-independent transformation starved of IL-3 overnight (IL3—in the figure indicates transformed cells). Cell lysates were immunoblotted for CSF3R, TNK2, phospho-STAT3 (pSTAT3), total STAT3, phospho-JAK2 (pJAK2), total JAK2, phospho-SRC (pSRC), total SRC and actin. Parental Ba/F3 cells or Ba/F3 cells expressing WT CSF3R (WT) are included as controls.

FIGS. 2A-2D collectively show that dysregulated signaling induced by CSF3R mutations may be targeted by tyrosine kinase inhibitors

FIG. 2A is a bar graph depicting the number of colonies formed by mouse bone marrow cells infected with mutant CSF3R-containing retroviruses, or an empty vector. Control cells therefore expressed endogenous WT CSF3R (Vector/endogenous). Cells were grown in methylcellulose containing the minimal amount of GCSF necessary to form colonies (Vector/endogenous—10 ng/mL, S783fs—0.4 ng/mL, and T618I—no GCSF). Cells were plated with increasing concentrations of dasatinib (0, 1, 10, 100 and 1000 nM). The experiment was performed in triplicate with the number of colonies normalized to the untreated controls. Values represent mean percent colonies±s.e.m. * p<0.07; ** p<0.005 when comparing CSF3R T618I to S783fs at equivalent doses of dasatinib.

FIG. 2B is a bar graph depicting the result of a similar colony formation assay was performed as in FIG. 2A except that cells were plated with 0, 10, 100 or 1000 nM ruxolitinib. Values represent mean percent colonies±s.e.m.

FIG. 2C is a line graph of the response of CNL patient #9 to ruxolitinib. Patient #9 was shown to be sensitive to ruxolitinib (rux) in vitro and harbored a T618I mutation (FIG. S3C, FIG. S3E). This patient was treated with 500 mg hydroxyurea (HU; hydroxycarbamide) daily starting on day 13. Hydroxyurea was stopped on day 21 and 10 mg ruxolitinib taken orally twice daily was initiated. On day 70 the dose of ruxolitinib was escalated to 15 mg twice daily. White blood cells (black line, WBC) and absolute neutrophil count (gray line, ANC) are shown.

FIG. 2D is a line graph showing the ruxolitinib normalized platelet counts from patient #9 for the same time period described in FIG. 2C.

FIG. 3 is a model for the activation and signaling of CSF3R mutations.

Without being bound by theory, truncation mutations of CSF3R result in increased expression levels. Downstream signaling mediators, SRC-family kinases (SFKs) and TNK2, are preferentially activated by truncation mutant CSF3R. Consequently, leukemic cells harboring truncation mutant CSF3R are highly sensitive to dasatinib. Truncation mutant CSF3R may also exhibit sensitivity to JAK kinase inhibitors in the context of JAK kinase stimulation downstream of high ligand concentrations. In contrast, membrane proximal mutant CSF3R exhibits complete ligand independent function. In this capacity, the dominant mode of signaling appears to operate through the JAK/STAT pathway. Hence, patients exhibiting membrane proximal mutations may be candidates for JAK kinase inhibitors, such as the JAK1/2 inhibitor ruxolitinib.

FIG. 4 is a set of two images of samples from patient #10 in tables S3A and S3B below. The left panel is an image of peripheral blood showing segmented and band neutrophils with toxic granulation and Döhle bodies (arrow). These are common morphologic features among CNL and aCML patients carrying CSF3R mutations (Wright Giemsa stain, 1000×). The right panel is a bone marrow aspirate from the same patient. This reveals a marked myeloid hyperplasia with full spectrum maturation (Wright Giemsa stain, 400×).

FIGS. 5A-5C collectively show the results of deep sequencing and Sanger validation for patient #3.

FIG. 5A is an electropherogram Sanger sequence of DNA flanking the 1 bp insertion in CSF3R from patient #3 that was amplified and cloned into plasmids such that single clones could be sequenced to confirm the presence of the 1 bp insertion. Representative electropherograms of clones exhibiting the WT and S783fs alleles are shown.

FIG. 5B is an electropherogram of direct Sanger sequencing of amplified genomic DNA from Patient #3 showing that the S783fs mutation is present at nearly 50% frequency,

FIG. 5C is an electropherogram of direct Sanger sequencing of amplified genomic DNA from patient #3 showing the T615A mutation is a very low frequency mutation. This is further supported by cloning and enumeration of sequenced PCR products from this same specimen as described in Table 4. In all electropherograms, A is denoted as a green line, C as a blue line, G as a black line and T as a red line.

FIGS. 6A-6D collectively show deep sequencing and Sanger validation for T618I-positive ETP-T-ALL and CNL patient #9

FIG. 6A is an electropherogram from Sanger sequencing of the patient in FIG. S3A revealing a C to T mutation resulting in a T618I mutation in CSF3R. In the electropherogram, A is denoted as a green line, C as a blue line, G as a black line and T as a red line.

FIG. 6B is an electropherogram from Sanger Sequencing of the CNL patient #9 that revealed a T618I point mutation in CSF3R. A single base pair mutation (C to T) was found at approximately 50% frequency and the electropherograms for the region surrounding the mutation are shown.

FIG. 6C is a line graph of cells from CNL patient #9 tested for sensitivity to dasatinib. The dasatinib curves were run in triplicate and did not reach an IC₅₀, even at the highest concentration tested (1000 nM). Values represent mean percent cell viability±s.e.m.

FIG. 6D is a line graph from the same CNL patient sample (patient #9) tested for sensitivity to ruxolitinib. The patient specimen was sensitive to ruxolitinib with an IC₅₀ of 127 nM. Values represent mean percent cell viability.

### SEQUENCE LISTING

SEQ. ID NO: 1 is the amino acid sequence of CSF3R.

SEQ. ID NO: 2 is a PCR primer to amplify exon 14 of CSF3R.

SEQ. ID NO: 3 is a PCR primer to amplify exon 14 of CSF3R.

SEQ. ID NO: 4 is a PCR primer to amplify exon 17 of CSF3R.

SEQ. ID NO: 5 is a PCR primer to amplify exon 17 of CSF3R.

SEQ. ID NO: 6 is an M13 forward primer.

SEQ. ID NO: 7 is an M13 reverse primer.

SEQ. ID NO: 8 is the nucleic acid sequence of Exon 14 of CSF3R.

SEQ. ID NO: 9 is the nucleic acid sequence of Exon 17 of CSF3R.

### DETAILED DESCRIPTION

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell 10 Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

In addition, the materials, methods, and examples are illustrative only and not intended to be limiting. In order to facilitate review of the various embodiments of the disclosure, the following explanations of specific terms are provided:

Aberrant activity: Activity of any protein that results in inappropriate or uncontrolled activation of a tyrosine kinase, such as a tyrosine kinase that acts downstream of CSF3R (such as JAK, STAT, SRC, TNK2 and/or LCK, for example by over-expression, upstream activation (for example, by aberrant upstream activation of the tyrosine kinase by a mutant form of CSF3R), and/or mutation (for example a truncation, deletion, insertion and/translocation which increases the activity, such as but not limited to, kinase activity of a tyrosine kinase), which can lead to uncontrolled cell growth, for example in hematological malignancies such as CNL or aCML. In some examples, aberrant activity of a tyrosine kinase is a higher rate of kinase activity than the rate of kinase activity in a cell that has a wild-type CSF3R.

Administration: To provide or give a subject an agent, such as a composition that targets/inhibits a tyrosine kinase acting downstream of a mutant CSF3R (such as dasatinib or a JAK inhibitor) by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Amplifying a nucleic acid molecule: To increase the number of copies of a nucleic acid molecule, such as a gene or fragment of a gene, for example a region of a gene that encodes a tumor biomarker, such as exons 14 and/or 17 of CSF3R. The resulting products are called amplification products. An example of in vitro amplification is the polymerase chain reaction (PCR). Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. SYBR green binds to double stranded DNA, but not to single stranded DNA. In addition, SYBR green fluoresces strongly at a wavelength of 497 nm when it is bound to double stranded DNA, but does not fluoresce when it is not bound to double stranded DNA. As a result, the intensity of fluorescence at 497 nm may be correlated with the amount of amplification product present at any time during the reaction. The rate of amplification may in turn be correlated with the amount of template sequence present in the initial sample. Generally, Ct values are calculated similarly to those calculated using the TaqMan® system. Because the probe is absent, amplification of the proper sequence may be checked by any of a number of techniques. One such technique involves running the amplification products on an agarose or other gel appropriate for resolving nucleic acid fragments and comparing the amplification products from the quantitative real time PCR reaction with control DNA fragments of known size.

Another commonly used method is real-time quantitative TaqMan® PCR (Applied Biosystems). This type of PCR has reduced the variability traditionally associated with quantitative PCR, thus allowing the routine and reliable quantification of PCR products to produce sensitive, accurate, and reproducible measurements of levels of gene expression. The PCR step can use any of a number of thermostable DNA-dependent DNA polymerases, it typically employs a Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used.

Two oligonucleotide primers are used to generate an amplicon typical of a PCR reaction. A third oligonucleotide, or probe, is designed to detect nucleotide sequence located between the two PCR primers. The probe is nonextendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Examples of fluorescent labels that may be used in quantitative PCR include but need not be limited to: HEX, TET, 6-FAM, JOE, Cy3, Cy5, ROX TAMRA, and Texas Red. Examples of quenchers that may be used in quantitative PCR include, but need not be limited to TAMRA (which may be used as a quencher with HEX, TET, or 6-FAM), BHQ1, BHQ2, or DABCYL. TAQMAN® RT-PCR can be performed using commercially available equipment, such as, for example, ABI PRISM 7700® Sequence Detection System (Perkin-Elmer—Applied Biosystems), or Lightcycler (Roche Molecular Biochemicals).

In one embodiment, the 5′ nuclease procedure is run on a real-time quantitative PCR device such as the ABI PRISM 7700® Sequence Detection System. The system includes a thermocycler, laser, charge-coupled device (CCD), camera and computer. The system amplifies samples in a 96-well format on a thermocycler. During amplification, laser-induced fluorescent signal is collected in real time through fiber optic cables for all 96 wells, and detected at the CCD. The system includes software for running the instrument and for analyzing the data. In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR can be performed using an internal standard. The ideal internal standard is expressed at a constant level among different tissues, and is unaffected by the experimental treatment. RNAs most frequently used to normalize patterns of gene expression are the mRNA products of housekeeping genes.

Amplification of a nucleic acid sequence may be used for any of a number of purposes, including increasing the amount of a rare sequence to be analyzed by other methods. It may also be used to identify a sequence directly (for example, though an amplification refractory mutation system) or as part of a DNA sequencing method.

Antibody: A polypeptide including at least a light chain or heavy chain immunoglobulin variable region which specifically recognizes and binds an epitope of an antigen (such as a mutant CSF3R) or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, heteroconjugate antibodies (such as, bispecific antibodies). See also, Pierce Catalog and Handbook, 1994-1995 (Pierce Chemical Co., Rockford, Ill.); Kuby, J., Immunology, 3rd Ed.,W.H. Freeman & Co., New York, 1997.

Anti-proliferative activity: An activity of a molecule, for example a small molecule, an inhibitory RNA, and the like, which reduces proliferation of at least one cell type, but which may reduce the proliferation (either in absolute terms or in rate terms) of multiple different cell types (e.g., different cell lines, different species, etc.). In specific embodiments, the anti-proliferative activity of a small molecule, such as an inhibitor of a downstream effector of a mutant CSF3R will be apparent against cancer cells obtained from a subject that has aberrant CSF3R activity, including cells that have aberrant CSF3R activity and one or more mutations that render the cancer susceptible to dasatanib an/or JAK inhibitors.

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. In certain example arrays, one or more molecules (such as an antibody or peptide) will occur on the array a plurality of times (such as twice), for instance to provide internal controls. The number of addressable locations on the array can vary, for example from at least one, to at least 2, to at least 3, at least 4, at least 5, at least 6, at least 10, at least 20, at least 30, at least 50, at least 75, at least 100, at least 150, at least 200, at least 300, at least 500, least 550, at least 600, at least 800, at least 1000, at least 10,000, or more. In some examples, arrays include positive and/or negative controls, such as probes that bind housekeeping genes. In particular examples, an array includes nucleic acid molecules, such as oligonucleotide sequences that are at least 15 nucleotides in length, such as about 15-75 or 15-60 nucleotides in length. In particular examples, an array includes oligonucleotide probes or primers which can be used to detect nucleotides that encode tumor biomarker sequences (including mutant forms of CSF3R). In an example, the array is a commercially available array such as Human Genome GeneChip® arrays from Affymetrix (Santa Clara, Calif.).

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays may be computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Binding or stable binding: An association between two substances or molecules, such as the association of an antibody with a peptide, nucleic acid to another nucleic acid, or the association of a protein with another protein or nucleic acid molecule, or the association of a small molecule drug with a protein (such as a tyrosine kinase) or other biological macromolecule. Binding can be detected by any procedure known to one skilled in the art, such as by physical or functional properties. For example, binding can be detected functionally by determining whether binding has an observable effect upon a biosynthetic process such as expression of a gene, DNA replication, transcription, translation, protein activity (including tyrosine kinase activity) and the like.

Biological signaling pathway: A systems of proteins including cell surface receptors and their downstream effector molecules such as tyrosine kinases and other molecules that act in an orchestrated fashion to mediate the response of a cell toward internal and external signals. In some examples, biological signaling pathways include downstream effector kinases such as SRC kinases, TNK2, JAK kinases, STAT kinases and others which can propagate signals in the pathway by selectively phosphorylating downstream substrates. In some examples a biological signaling pathway is disregulated and functions improperly, which can lead to aberrant signaling and in some instances hyper-proliferation of the cells with the aberrant signaling. In some examples, disregulation of a biological signaling pathway can result in a malignancy, such as cancer, for example the aberrant activation of CSF3R by one or more mutations in exon 14 and/or exon 17 such as D771fs, G683R, S783fs, T615A, T618I, Y752X, E808K, or W791X.

Biomarker: Molecular, biological or physical attributes that characterize a physiological or cellular state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination.

A biomarker can be represented by the sequence of a nucleic acid from which it can be derived or any other chemical structure. Examples of such nucleic acids include miRNA, tRNA, siRNA, mRNA, cDNA, or genomic DNA sequences including any complimentary sequences thereof. One example of a biomarker is a DNA coding sequence for a protein comprising one or more mutations that cause amino acid substitutions in the protein sequence. Such a biomarker may be the coding sequence of a particular part of a protein such as exon 14 and/or exon 17 of CSF3R comprising nucleic acid mutations that cause one or more of the following amino acid substitutions: D771fs, G683R, S783fs, T615A, T618I, Y752X, E808K, or W791X.

Cancer: A disease or condition in which abnormal cells divide without control and are able to invade other tissues. Cancer cells spread to other body parts through the blood and lymphatic systems. Cancer is a term for many diseases. There are more than 100 different types of cancer in humans. Most cancers are named after the organ in which they originate. For instance, a cancer that begins in the colon may be called a colon cancer. However, the characteristics of a cancer, especially with regard to the sensitivity of the cancer to therapeutic compounds, are not limited to the organ in which the cancer originates. A cancer cell is any cell derived from any cancer, whether in vitro or in vivo.

Cancer is a malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

“Metastatic disease” or “metastasis” refers to cancer cells that have left the original tumor site and migrate to other parts of the body for example via the bloodstream or lymph system. The “pathology” of cancer includes all phenomena that compromise the wellbeing of the subject. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

Chemotherapeutic agent or Chemotherapy: Any chemical agent with therapeutic usefulness in the treatment of diseases characterized by abnormal cell growth. Such diseases include tumors, neoplasms, and cancer as well as diseases characterized by hyperplastic growth. In one embodiment, a chemotherapeutic agent is an agent of use in treating cancer, such as cancers characterized by aberrant CSF3R activity, including cancers characterized by aberrant CSF3R activity comprising mutations exons 14 and 17. Such agents include inhibitors of downstream effector molecules of CSF3R such as dasatinib and JAK kinase inhibitors such as ruxolitinib. Combination chemotherapy is the administration of more than one agent to treat cancer.

Contacting: Placement in direct physical association, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Dasatinib: the generic name of N-(2-chloro-6-methylphenyl)-2-[[6-[4-(2-hydroxyethyl)-1-piperazinyl]-2-methyl-4-pyrimidinyl]amino]-5-thiazole carboxamide monohydrate. Dasatinib is sold under the trade name Sprycel® by Bristol-Myers Squibb. Dasatinib is a tyrosine kinase inhibitor that inhibits a large number of aberrantly active tyrosine kinases and can be used to treat cancer characterized by aberrant activity of any one or combination of those tyrosine kinases. Examples of tyrosine kinases inhibited by dasatinib include: ABL, ARG, BCR-ABL, KIT, PDGFR, SRC, YES, FYN, LYN, HCK, LCK, FGR, BLK, FRK, CSK, BTK, TEC, BMX, TXK, DDR1, DDR2, ACK, ACTR2B, ACVR2, BRAF, EGFR/ERBB1, EPHA2, EPHA3, EPHA4, EPHA5, EPHA8, EPHB1, EPHB2, EPHB4, EPHB6, ERBB2, ERBB4, FAK, GAK, GCK, HH498/TNNI3K, ILK, LIMK1, LIMK2, MAP2K5, MAP3K1, MAP3K2, MAP3K3, MAP3K4, MAP4K1, MAP4K5/KHS1, MAPK11/p38 beta, MAPK14/p38 alpha, MYT1, NLK, PTK6/Brk, QIK, QSK, RAF1, RET, RIPK2, SLK, STK36/ULK, SYK, TAO3, TESK2, TYK2, and ZAK (Hantschel O et al, Leukemia and Lymphoma 49, 615-619 (2008); incorporated by reference herein.)

Diagnostic: Identifying the presence or nature of a pathologic condition, such as, but not limited to cancer, aCML, CNL, or any cancer caused by aberrant CSF3R activity, including cancer caused by aberrant CSF3R activity that is sensitive to dasatinib and/or JAK kinase inhibitors such as ruxolitinib. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis.

Domain: any part of polypeptide that can be demonstrated to mediate a particular protein function.

Effective amount: An amount of agent, such as a tyrosine kinase inhibitor that is sufficient to generate a desired response, such as reduce or eliminate a sign or symptom of a condition or disease, such as cancer, for example cancers characterized by aberrant expression of CSF3R. When administered to a subject, a dosage will generally be used that will achieve target tissue concentrations that has been shown to achieve anti proliferative activity in vitro. In some examples, an “effective amount” is one that treats (including prophylaxis) one or more symptoms and/or underlying causes of any of a disorder or disease, for example cancer, such as a cancer characterized by an aberrant CSF3R. An effective amount can be a therapeutically effective amount, including an amount that prevents one or more signs or symptoms of a particular disease or condition from developing, such as one or more signs or symptoms associated with cancer.

Inhibitor: Any chemical compound, specific for a protein or other gene product that can directly interfere with the activity of a protein, such as a kinase, particularly a kinase that is downstream effector molecule of CSF3R, and most particularly a kinase that is a downstream effector molecule of a CSF3R mutant comprising one or more of the following mutations: D771fs, G683R, S783fs, T615A, T618I, Y752X, E808K, and W791X. An inhibitor can inhibit the activity of a protein either directly or indirectly. Direct inhibition can be accomplished, for example, by binding to a protein and thereby preventing the protein from binding an intended target, such as a receptor. Indirect inhibition can be accomplished, for example, by binding to a protein's intended target, such as a receptor or binding partner, thereby blocking or reducing activity of the protein.

Inhibit: To reduce to a measurable extent, for example, to reduce activity (including aberrant activity) of a protein such as a kinase. In some examples, the kinase activity of a protein is inhibited, for example using a small molecule inhibitor of a downstream effector of CSF3R such as dasatinib or a JAK kinase inhibitor such as ruxolitinib.

Inhibiting or treating a disease: Inhibiting the full development of a disease or condition, for example, in a subject who has or who is at risk for a disease such cancer, for example, CNL, aCML, or any cancer characterized by aberrant CSF3R activity. “Treatment” refers to any intervention that ameliorates a sign or symptom of a disease or pathological condition before or after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A prophylactic treatment is a treatment administered to a subject who does not exhibit signs of a disease or exhibits only early signs for the purpose of decreasing the risk of developing pathology. A therapeutic treatment is a treatment administered to a subject that has already begun exhibiting signs of a disease, particularly pathological signs.

Kinase: An enzyme that catalyzes the transfer of a phosphate group from one molecule to another. Kinases play a role in the regulation of cell proliferation, differentiation, metabolism, migration, and survival. A “tyrosine kinase” transfers phosphate groups to a hydroxyl group of a tyrosine in a polypeptide. In some examples, the kinase is a downstream effector molecule of CSF3R such as a JAK kinase, a Src family kinase or TNK2. Receptor protein tyrosine kinases (PTKs) contain a single polypeptide chain with a transmembrane segment. The extracellular end of this segment contains a high affinity ligand-binding domain, while the cytoplasmic end comprises the catalytic core and the regulatory sequences.

Mass spectrometry: A method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation.

Mutation: A mutation is any difference in a nucleic acid or polypeptide sequence from a normal, consensus or “wild type” sequence. A mutant is any protein or nucleic acid sequence comprising a mutation. In addition a cell or an organism with a mutation may also be referred to as a mutant.

Some types of mutations include point mutations (differences in individual nucleotides or amino acids); silent mutations (differences in nucleotides that do not result in an amino acid changes); deletions (differences in which one or more nucleotides or amino acids are missing); frameshift mutations (differences in which deletion of a number of nucleotides indivisible by 3 results in an alteration of the amino acid sequence. Frameshift mutations may be described by the point at which the frameshift begins. A mutation that results in a difference in an amino acid may also be called an amino acid substitution mutation. Amino acid substitution mutations may be described by the amino acid change relative to wild type at a particular position in the amino acid sequence. Amino acid substitution mutations that result in a mutation from an amino acid residue to a stop codon (a protein truncation) can be described by identifying the residue which is mutated followed by an X. Examples of such mutations in CSF3R include D771fs, G683R, S783fs, T615A, T618I, Y752X, E808K, and W791X. D771F refers to a frameshift mutation beginning at the aspartic acid at position 771. T615A refers to a threonine to alanine amino acid substitution at position 615. T618I refers to a threonine to isoleucine amino acid substitution at position 618. W791X refers to a mutation from a tryptophan to a stop codon at position 791.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phospho-peptide or phospho-protein: A protein in which one or more phosphate moieties are covalently linked to one or more of the amino acids making up the peptide or protein. A peptide can be phosphorylated at multiple or single sites. Sometimes it is desirable for the phospho-protein to be phosphorylated at one site regardless of the presence of multiple potential phosphorylation sites. In vivo the transfer of a phosphate to a peptide is accomplished by a kinase. For example a tyrosine kinase such as a JAK kinase, a SRC kinase, or TNK2 transfers a phosphate to a tyrosine residue of a substrate peptide or protein.

Polypeptide: Any chain of amino acids, regardless of length or posttranslational modification (such as glycosylation, methylation, ubiquitination, phosphorylation, or the like). In one embodiment, a polypeptide is CSF3R. “Polypeptide” is used interchangeably with “protein,” and is used to refer to a polymer of amino acid residues. A “residue” refers to an amino acid or amino acid mimetic incorporated in a polypeptide by an amide bond or amide bond mimetic.

Purified: The term “purified” does not require absolute purity; rather, it is intended as a relative term. Thus, for example, a purified nucleic acid preparation is one in which the protein referred to is more pure than the protein in its natural environment within a cell. For example, a preparation of a protein is purified such that the protein represents at least 50% of the total protein content of the preparation. Similarly, a purified nucleic acid preparation is one in which nucleic acid is more pure than in an environment including a complex mixture compounds, such as a cell or cell extract. The terms “isolated” or “isolating” are interchangeable with the term “purified” or “purifying.”

Sample: A sample, such as a biological sample, is a sample obtained from a plant or animal subject. As used herein, biological samples include all clinical samples useful for detection of mutations in tumor DNA, particularly mutations in exon 14 or exon 17 of CSF3R. Samples include, but not limited to, cells, tissues, and bodily fluids, including tissues that are, for example, unfixed, frozen, fixed in formalin and/or embedded in paraffin. In one example, a sample includes a tissue biopsy obtained from a subject with a tumor.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Small molecule: A molecule, typically with a molecular weight less than about 1000 Daltons, or in some embodiments, less than about 500 Daltons, wherein the molecule is capable of modulating, to some measurable extent, an activity of a target molecule such as inhibiting the activity of a kinase, such as a downstream effector molecule of a CSF3R polypeptide with aberrant activity.

Subject: A living multicellular vertebrate organism, a category that includes, for example, mammals and birds. A “mammal” includes both human and non-human mammals, such as mice. In some examples, a subject is a patient, such as a patient diagnosed with a hematological cancer.

Substrate: A molecule that is acted upon by an enzyme. A substrate binds with the enzyme's active site, and an enzyme-substrate complex is formed. In some examples, the enzyme catalyses the incorporation of an atom or other molecule into the substrate, for example a kinase can incorporate a phosphate into the substrate, such as a peptide, thus forming a phospho-substrate.

Tissue: A plurality of functionally related cells. A tissue can be a suspension, a semi-solid, or solid. Tissue includes parts of organs collected from a subject such as the lung, the liver or a portion thereof.

### Methods of Diagnosis

Disclosed herein are methods of diagnosing aCML or CNL in a subject. In particular examples, the methods include identifying a nucleic acid mutation in CSF3R (SEQ ID NO: 1), including a nucleic acid mutation in exon 14 of CSF3R (SEQ ID NO: 8) or exon 17 of CSF3R (SEQ ID NO: 9) of that results in one of the following amino acid substitutions: D771fs, G683R, S783fs, T615A, T618I, Y752X, E808K, or W791X either alone or in combination. The disclosed mutations may be identified by any suitable method known in the art. For example, they may be detected by any method of nucleic acid sequencing, through any method involving nucleic acid amplification, by any method of detecting a protein with one or more of the disclosed mutations, or any combination thereof. Examples of these methods are discussed in detail below.

In some embodiments, the mutation is detected in a biological sample obtained from the subject. Biological samples include cancer cells. For hematological malignancies, such biological samples can be taken from whole blood, from bone marrow aspirates, or any other source of tissue that could contain the hematological malignancy. Tumor samples can also include normal tissue. This normal tissue may serve as an internal negative control, especially in the case of assays that detect the presence of a biomarker in the context of tissue structure, including immunohistochemistry, FACS analysis, or in situ hybridization. It will appreciated by those of skill in the art that any method of obtaining tissue from a subject can be utilized, and that the selection of the method used will depend upon various factors such as the type of tissue, age of the subject, or procedures available to the practitioner.

### Detecting Cancer Biomarkers

The disclosed CSF3R mutations can be detected in a sample using any one of a number of methods well known in the art. Nucleic acids such as genomic DNA, particularly tumor genomic DNA can be isolated from a tumor sample, such as, in the case of a hematological malignancy, whole blood collected from a subject. General methods of nucleic acid isolation are well known to those of skill in the art. Such methods are disclosed in standard textbooks and handbooks of molecular biology and embodied in commercially available kits.

The mutations can be detected through nucleic acid sequencing. Sequencing may be performed on genomic DNA from the tumor through any method known in the art including Sanger sequencing, pyrosequencing, SOLiD® sequencing, massively parallel sequencing, barcoded sequencing, or any other sequencing method now known or yet to be disclosed.

In Sanger Sequencing, a single-stranded DNA template, an oligonucleotide primer, a DNA polymerase, and nucleotides are used. A label, such as a radioactive label or a fluorescent label is conjugated to some of the nucleotides. One chain terminator base comprising a dideoxynucleotide (ddATP, ddGTP, ddCTP, or ddTTP, replaces the corresponding deoxynucleotide in each of four reactions. The products of the DNA polymerase reactions are electrophoresed and the sequence determined by comparing a gel with each of the four reactions. In another example of Sanger sequencing, each of the chain termination bases is labeled with a fluorescent label and each fluorescent label is of a different wavelength. This allows the polymerization reaction to be performed as a single reaction and enables greater automation of sequence reading.

In pyrosequencing, the addition of a base to a single stranded template to be sequenced by a polymerase results in the release of a pyrophosphate upon nucleotide incorporation. An ATP sulfyrlase enzyme converts pyrophosphate into ATP which in turn catalyzes the conversion of luciferin to oxyluciferin which results in the generation of visible light that is then detected by a camera.

In SOLiD® sequencing, the molecule to be sequenced is fragmented and used to prepare a population of clonal magnetic beads (in which each bead is conjugated to a plurality of copies of a single fragment) with an adaptor sequence. The beads are bound to a glass surface. Sequencing is then performed through 2-base encoding.

In massively parallel sequencing, randomly fragmented targeted DNA is attached to a surface through the use of an oligonucleotide adaptor. The fragments are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

In pyrosequencing, massively parallel sequencing or SOLiD® sequencing, an artificial sequence called a barcode may be added to primers used to clone fragmented sequences or to adaptor sequences. A barcode is a 4-10 nucleic acid sequence that uniquely identifies a sequence as being derived from a particular sample. Barcoding of samples allows sequencing of multiple samples in a single sequencing run. (See Craig D W et al, Nat Methods 5, 887-893 (2008) for descriptions and examples of barcodes.) DNA sequencing methods can, but need not, rely on nucleic acid amplification of a nucleic acid encoding a protein such CSF3R.

Additional methods of detecting mutations in nucleic acids include detection through selective nucleic acid amplification of mutant sequences. An example of such a method is the amplification refractory mutation system (ARMS) Newton et al, Nucleic Acids Res 17, 2503-2515 (1989.) This method uses a primer that matches the nucleotide sequence immediately 5′ of the mutation to be tested with the 3′ end of the primer specific for the nucleotide sequence of the mutant. Such a primer will specifically amplify the mutant nucleic acid but not the wild type amino acid. Such reactions may be adapted to real-time PCR based systems such as TaqMan®. The disclosed mutations may also be identified using a microarray technique. Sequences corresponding to one or more of the disclosed mutants may be plated or arrayed on a microchip substrate. The arrayed sequences are then hybridized to isolated tumor genomic DNA. An array may also be a multi well plate.

The disclosed mutations may also be identified in proteins by, for example, mass spectrometry or antibodies designed to detect proteins with the disclosed mutations.

### Methods of Treatment

Disclosed herein are methods of treating a cancer in a subject characterized by aberrant CSF3R activity, such as a CNL or aCML. The methods include selecting a subject with a tumor or tumor clone that has aberrant CSF3R activity, such as a subject CNL or aCML or a subject suspected of having CNL or aCML. The subject is then treated with a therapeutic agent such as dasatinib or a JAK kinase inhibitor such as ruxolitinib, particularly when the CSF3R comprises one or more of the following mutations: D771fs, G683R, S783fs, T615A, T618I, Y752X, E808K, or W791X including the homologs thereof for non-human mammals.

The administration of the therapeutic agent can be for either a prophylactic or a therapeutic purpose. When provided prophylactically, the therapeutic agent is provided in advance of any symptom. The prophylactic administration of the compounds serves to prevent or ameliorate any subsequent disease process. When provided therapeutically, the therapeutic agent is provided at (or shortly after) the onset of a symptom of disease. For prophylactic and therapeutic purposes, the therapeutic agent can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition. One of skill in the art in light of this disclosure will be able to determine an effective dose of dasatinib and/or a JAK kinase inhibitor such as ruxolitinib.

### Kits

A diagnostic kit may contain reagents such as oligonucleotides configured to perform nucleic acid amplification (including TaqMan® amplification) that specifically recognize mutant nucleic acids that cause amino acid changes such as D771fs, G683R, S783fs, T615A, Y752X, E808K, or W791X; in CSF3R. A diagnostic kit may also comprise an array that includes oligonucleotides that detect the disclosed mutations. A diagnostic kit may also contain a set of primers that amplify the kinase domain for sequencing or any other nucleic acid analysis. A diagnostic kit may also comprise antibodies specific for mutant forms of the CSF3R, including D771fs, G683R, S783fs, T615A, T618I, Y752X, E808K, and W791X.

### Examples

The following examples are illustrative of disclosed methods. In light of this disclosure, those of skill in the art will recognize that variations of these examples and other examples of the disclosed method would be possible without undue experimentation.

### Example 1

### Methods

Functional Analysis of Patient Samples:

Bone marrow or blood samples from patients with acute leukemia were separated using a Ficoll gradient followed by red blood cell lysis; specimens from patients with CNL or aCML were processed with red blood cell lysis only. Cells were cultured in RPMI-1640 medium (Invitrogen) containing 10% fetal bovine serum (FBS, Atlanta Biologicals), L-glutamine (Invitrogen), fungizone (Invitrogen), penicillin/streptomycin (Invitrogen), and 10⁻⁴ M 2-mercaptoethanol (Sigma). Screening of primary leukemia cells against panels of siRNA and kinase inhibitors was performed as previously described. Screening of primary leukemia cells against panels of siRNA and kinase inhibitors was performed as previously described (Bicocca V T et al, Cancer Cell 22, 656-667 (2012); Tyner J W et al, Proc Natl Acad Sci USA 106, 8695-8700 (2009); Tyner J W et al, Cancer Res 73, 285-296 (2013); and Tyner J W et al, Cancer Res 73, 285-296 (2013); all of which are incorporated by reference herein.)

Genomic Analysis of Patient Samples:

Genomic DNA was isolated from cryopreserved patient sample material using Qiagen DNeasy® columns. DNA was fragmented by sonication using an S2® Sonicator (Covaris). Fragmented DNA was then processed according to the SeqEZ® protocol (Nimblegen/Roche), which is based on the TruSeq® protocol (Illumina). Briefly, following fragmentation, DNA was blunt-ended and then 3′ tailed with a single “A” nucleotide. Fragments were ligated to adaptors containing indexed barcodes. The library was then size selected using a 2% agarose gel. The recovered library was amplified by limited rounds of polymerase chain reaction (PCR). Solution capture was performed using a custom DNA probe capture library. The library was hybridized to the probe set for 72 hours at 47° C. Captured DNA was then recovered from the probes and amplified by limited rounds of PCR. The amplified library was separated from unincorporated nucleotides and primers using a QIAquick® PCR purification column (Qiagen), followed by verification of the library using the Bioanalyzer® (Agilent). Library concentration was determined using real time PCR on a StepOne® Real Time System (Life Technologies). The libraries were sequenced on a HiSeq 2000® sequencer (Illumina) followed by FASTQ assembly using the CASAVA pipeline (Illumina). Sequence capture, library preparation, and deep sequencing were performed by the OHSU Massively Parallel Sequencing Shared Resource.

CSF3R mutations were confirmed by PCR amplification of CSF3R exons using M13-tagged primers:

(SEQ ID NO: 2)
Exon 14 Forward -
GTAAAACGACGGCCAGTCCACGGAGGCAGCTTTAC
(SEQ ID NO: 3)
Exon 14 Reverse -
CAGGAAACAGCTATGACCAAATCAGCATCCTTTGGGTG
(SEQ ID NO: 4)
Exon 17 Forward -
GTAAAACGACGGCCAGTAGTGGCCCAAAGACACAGTC
(SEQ ID NO: 5)
Exon 17 Reverse -
CAGGAAACAGCTATGACCGGGAGTCCCATAACAGCTCA

PCR amplification was followed by purification of PCR products using Amicon® Ultra 0.5 mL 30K Centrifugal Filters (Millipore) and Sanger sequencing with M13 forward (GTAAAACGACGGCCAGT) (SEQ ID NO: 7) and reverse (CAGGAAACAGCTATGACC) (SEQ ID NO: 8) primers. Sanger sequencing was performed by the OHSU DNA Sequence Analysis Shared Resource. Additional sequencing of CSF3R exons 14-17 was performed by Genewiz, Inc (South Plainfield, N.J.).

Alignment and Analysis of Deep Sequencing:

Reads were aligned using the BWA realignment algorithm (Li H and Durbin R Bioinformatics 25, 1754-1760 (2009); incorporated by reference herein) and genotyping was performed using the GATK toolkit (McKenna A et al, Genome Res 20, 1297-1303 (2010); incorporated by reference herein) following the ‘Best Protocol’ of the ‘Best Practice Variant Detection with the GATK v2’ (http://www.broadinstitute.oregatk/). Briefly, genotyping was performed after removing duplicate reads, local realignment around the 1000 Genome's indels and indels inferred from the gapped alignments of all samples (indels found in one sample helped inform the rest). Finally, the quality scores were recalibrated after excluding positions known to vary in dbSNP132. Both single nucleotide variants (SNVs) and small indels were detected. The effect of the variants was initially assessed relative to Ensembl build 56 gene models (Flicek P et al, Nucleic Acids Res 40, D84-D90 (2012), incorporated by reference herein).

Vectors and Cloning:

CSF3R transcript variant 1 (NM_000760.2) (incorporated by reference herein) pDONR vector was purchased from GeneCopoeia. CSF3R mutations were introduced using the QuikChange II XL® site-directed mutagenesis kit (Agilent Technologies). CSF3R was cloned into the MSCV-IRES-green fluorescent protein (GFP) plasmid using the Gateway® Cloning System (Invitrogen).

Cell Culture and Retrovirus Generation:

293T17 cells were obtained from American Type Culture Collection (ATCC) and grown in Dulbecco's Modified Eagle's Medium (DMEM) (Invitrogen) containing 10% FBS, L-glutamine, fungizone and penicillin-streptomycin. 293T17 cells were transfected with FuGENE 6® (Promega). To generate murine retrovirus, 293T17 cells were transfected with MSCV-IRES-GFP constructs and EcoPac helper plasmid. Ba/F3 cells were obtained from ATCC and grown in RPMI 1640 medium with 10% FBS, L-glutamine, fungizone, penicillin-streptomycin, and 15% WEHI-conditioned medium (a source of IL-3). Stable Ba/F3 cell lines were generated by infection of 3×10⁶ cells with 1 mL of murine retrovirus expressing WT CSF3R, CSF3R mutants or an empty control vector, followed by sorting GFP positive cells by FACSaria® (BD Biosciences).

Ba/F3 Transformation Assays:

Parental Ba/F3 cells or those stably expressing WT CSF3R or CSF3R mutants were washed three times and re-suspended in RPMI 1640 with 10% FBS, L-glutamine, fungizone and penicillin/streptomycin at a density of 5×10⁵ cells per mL. Viable cell counts were obtained using propidium iodide exclusion on a Guava® Personal Cell Analysis System (Millipore).

Immunoblotting:

Ba/F3 cells stably expressing CSF3R constructs were lysed in Cell Lysis Buffer (Cell Signaling) containing Complete Mini Protease Inhibitor Cocktail Tablets (Roche), Phosphatase Inhibitor Cocktail 2 (Sigma) and Phenylmethanesulfonyl fluoride solution (Sigma) and then quantitated using the Pierce BCA Protein Assay Kit (Thermo Scientific). Lysates were diluted in sample buffer (75 mM Tris pH 6.8, 3% SCS, 15% glycerol, 8% β-mercaptoethanol, 0.1% bromophenol blue). Lysates were then separated by SDS-PAGE and transferred to polyvinylidene fluoride (PVDF) membrane (Millipore) and subjected to immunoblot analysis using the following antibodies: anti-GCSF Receptor (CSF3R) antibody (abcam, ab126167), anti-ACK1 (TNK2) antibody (abcam, ab74091), Phospho-STAT3 (Tyr705) (Cell Signaling Technologies (CST), 9131), Stat3 Antibody (CST, 9132), Phospho-Jak2 (Tyr1007/1008) Antibody (CST, 3771), JAK2 (D2E12) XP Rabbit mAb (CST, 3230), Phospho-Src Family (Tyr416) Antibody (CST, 2101), Src Antibody (CST, 2108), Anti-Actin (Ab-1) Mouse mAb (JLA20) (Calbiochem, CP01). All primary antibodies were used at a 1:1000 dilution, except for Actin, which was used at a 1:5000 dilution. Anti-mouse or anti-rabbit IgG HRP conjugate secondary antibodies (Promega) were used at a 1:5000 dilution and immunoblots were developed using SuperSignal West Pico Chemiluminescent Substrate (Thermo Scientific) on a Lummi Imager (Boehringer Mannheim).

Murine Hematopoietic Colony Formation Assays:

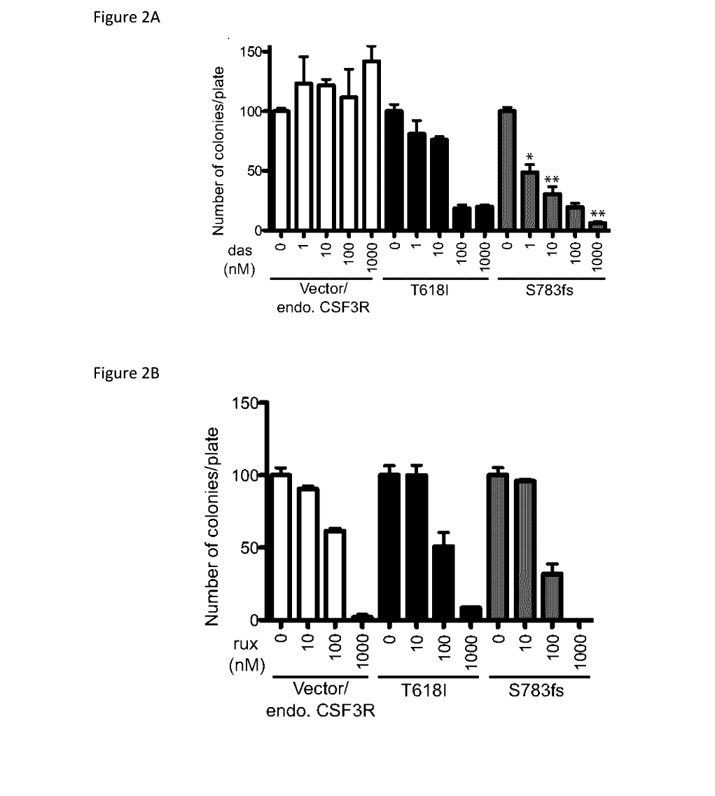

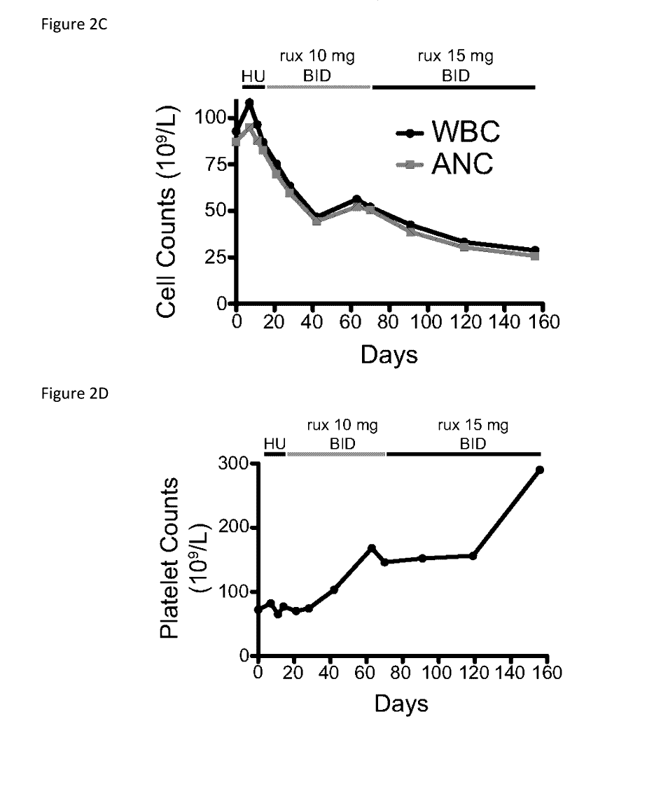

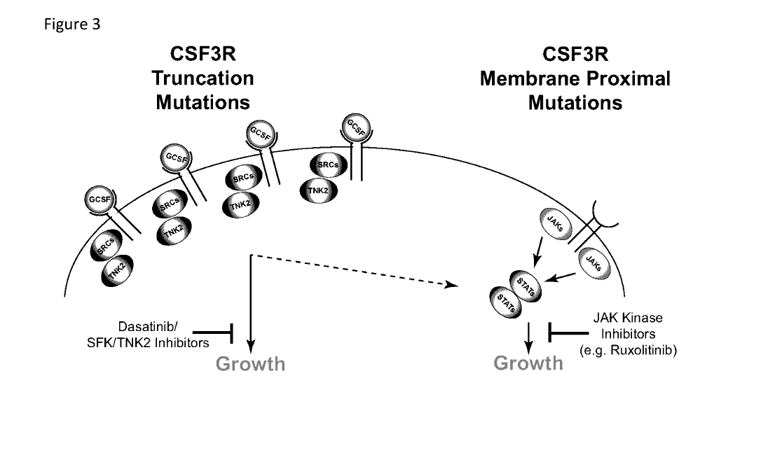

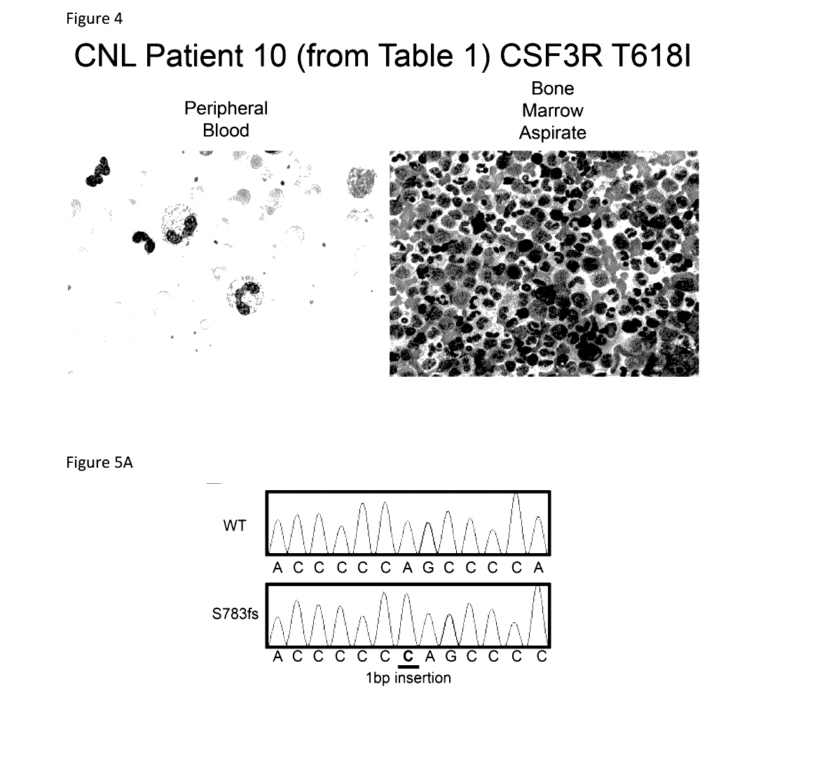

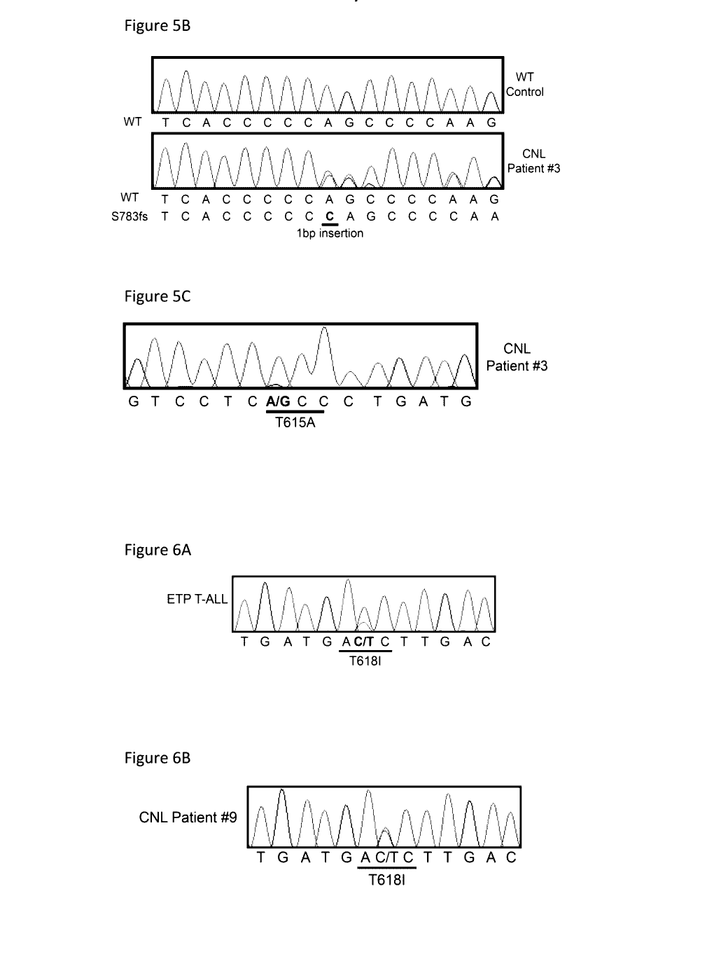

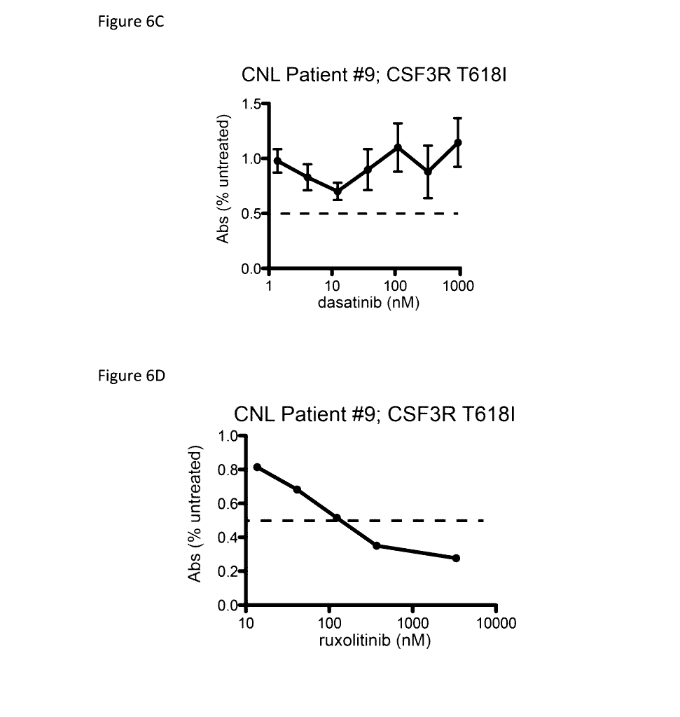

All mouse work was performed according to a protocol approved by the OHSU Institutional Animal Care and Use Committee. Bone marrow was isolated from 6-10 week old BALB/c mice (The Jackson Laboratory), red blood cells were lysed using ammonium chloride solution (Stem Cell Technologies), cells were then washed and incubated overnight in pre-stimulation medium, DMEM, 10% FBS, L-glutamine, penicillin/streptomycin, 15% WEHI conditioned medium, 7 ng/mL recombinant murine IL-3 (Peprotech), 12 ng/mL recombinant murine IL-6 (Peprotech) and 56 ng/mL recombinant murine SCF (Peprotech). Cells were infected on two successive days with murine retrovirus expressing WT CSF3R, CSF3R S783fs, CSF3R T618I or an empty vector control. Four hours after the second infection, cells were washed three times and 2.5×104 cells were plated in 1 mL of MethoCult® M3234 Methylcellulose Medium for Mouse Cells without cytokines (Stemcell Technologies) in triplicate. Recombinant human GCSF (Peprotech), dasatinib (LC laboratories) and ruxolitinib (Selleck Chemicals) were added to the medium prior to plating as described in the figures. Colony formation was scored manually by light microscopy one week after plating. Differences in response to dasatinib or ruxolitinib of CSF3R membrane proximal or truncation mutant colonies were evaluated for significance using a paired t-test with p-values as reported in the legend for FIG. 2.

### Example 2

### High Frequency of CSF3R Mutations in Patients with CNL/aCML

Patients with CNL/aCML may harbor oncogenes that would lead to small-molecule kinase inhibitor sensitivity. As such, a functional genomics approach was used to interrogate primary cells from 27 patients with CNL/aCML as well as specimens from patients with a variety of other hematologic malignancies. Deep sequencing with coverage of coding regions of 1,862 genes representing all kinases, phosphatases, non-kinase growth factor/cytokine receptors, and selected adaptor genes was also performed. Wherever possible, these primary leukemia cells were also screened against panels of small-molecule kinase inhibitors (Davis M I et al, Nat Biotechnol 29, 1046-1051 (2011); incorporated by reference herein) or tyrosine kinase-specific siRNAs Bicocca V T et al, Cancer Cell 22, 656-667 (2012); Tyner J W et al, Proc Natl Acad Sci USA 106, 8695-8700 (2009); and Tyner J W et al, Cancer Res 73, 285-296 (2013); all of which are incorporated by reference herein). As described in the cited references, this approach has been validated on specimens with proof-of-principle molecular lesions (e.g. BCR-ABL1, FLT3-ITD, JAK2 V617F, KRAS G13D) and has also been used to identify previously unknown molecular targets in leukemia specimens (e.g. MPL1886InsGG, ROR1).

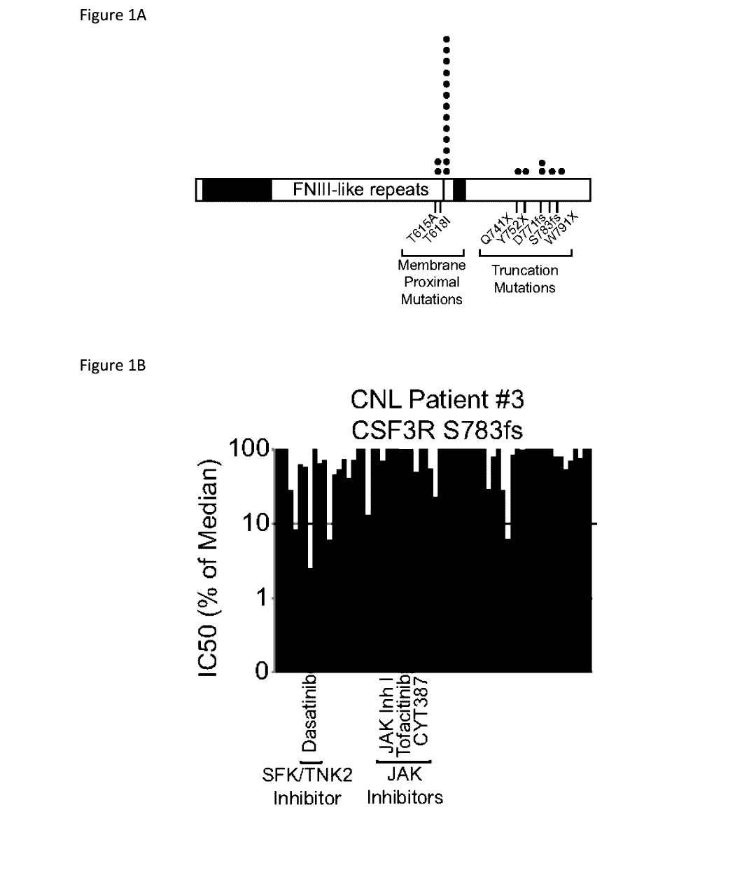

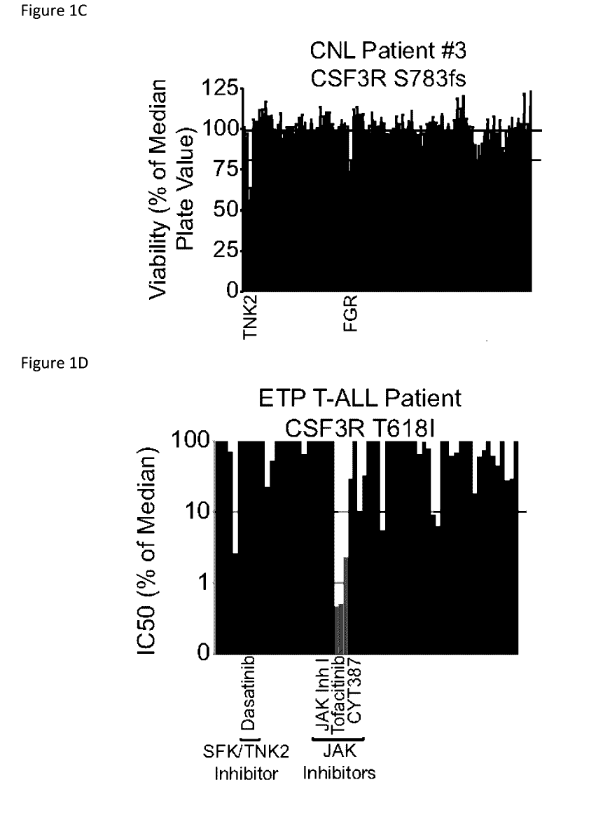

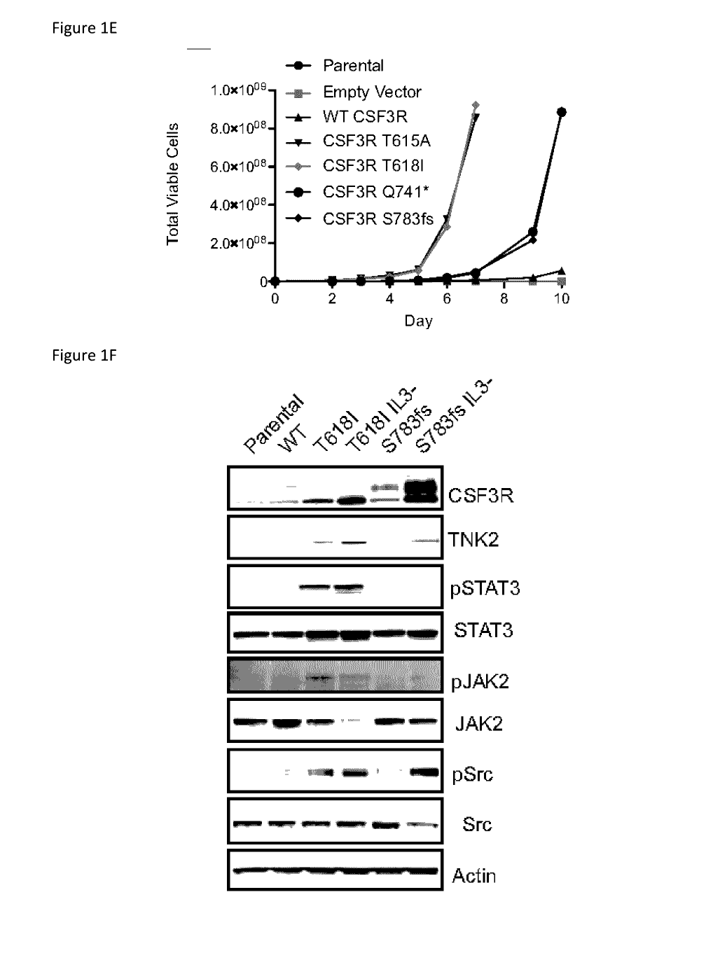

A significant enrichment of mutations in CSF3R was observed in patients with CNL/aCML (16/27 (59%); Table 1, Tables 2 and 3, FIG. 1A). Sequence variants identified included membrane proximal mutations (T615A, T618I) as well as a number of different frame-shift/nonsense mutations that truncate the cytoplasmic tail of CSF3R (truncation mutants: D771fs, S783fs, Y752X, W791X). Similar mutations that truncate the CSF3R cytoplasmic domain have been described in patients with congenital neutropenia who progress to AML after chronic GCSF treatment (Dong F, 1999 supra, Dong F, 1994 supra, and Germeshausen M, 2007 supra). Representative deep sequencing data and Sanger sequencing validation for cases with mutant CSF3R are shown in FIGS. 5A-5C and 6A-6D. Interestingly, five cases exhibited both membrane proximal and truncation mutations (Tables 2 and 3; Patients #3-7), and we confirmed that these compound mutations can occur on the same CSF3R allele with no requisite order for sequential acquisition of mutations (Table 4). In the setting of AML we identified 1/92 cases with at least CSF3R mutation; 2/200 CSF3R mutant AML cases have been reported within the Cancer Genome Atlas AML dataset indicating that the incidence of CSF3R mutations in AML is low (about 1%; Table 1). We identified one case of early T-cell precursor phenotype acute T-cell lymphoblastic leukemia (ETP-T-ALL) with CSF3R membrane proximal mutation (T618I; Table 1) from a total of 3 ETP-T-ALL cases. We found no additional CSF3R mutations in sequencing 8 other T-ALL cases or 41 B-ALL cases (Table 1). Finally, we sequenced three cases of reactive neutrophilia and none exhibited CSF3R mutations. Taken together, these data suggest that mutations within CSF3R are a defining molecular abnormality of CNL and aCML, and testing for CSF3R mutations could aid in the diagnosis of these diseases.

TABLE 1
Summary of CSF3R mutational status by hematologic
malignancy subset reveals enrichment of CSF3R
mutations in patients with CNL and aCML.

| | | Estimate of Disease |
|---|---|---|
| Diagnosis | Mutation | Frequency |
| CNL/aCML | 16/27 | 59% |
| AML | 3/292 | ~1% |
| T-ALL | 0/8 | 0 |
| ETP-T-ALL | 1/3 | unknown |
| B-ALL | 0/41 | 0 |

CNL—Chronic Neutrophilic Leukemia;
aCML—atypical Chronic Myeloid Leukemia,
BCR-ABL-negative;
AML—Acute Myeloid Leukemia;
T-ALL—T-cell Acute Lymphoblastic Leukemia;
ETP-T-ALL—Early T-cell Precursor T-ALL;
B-ALL—B-cell Acute Lymphoblastic Leukemia

### Example 3

### Dependence on SRC Family/TNK2 or JAK Kinases in CSF3R Mutant Leukemia

It was asked whether specimens harboring mutant CSF3R exhibit in vitro sensitivity to inhibitors or siRNA directed against kinases that become dysregulated downstream of mutant CSF3R. Analysis of cells from a CNL patient with CSF3R S783fs (patient #3, Table 2 and Table 3; FIGS. 5A-5D) revealed dramatic sensitivity to the multi-kinase inhibitor dasatinib (Sprycel™, Bristol-Myers Squibb), but no sensitivity to inhibitors of JAK family kinases (FIG. 1B). Further interrogation with the panel of tyrosine kinase specific siRNAs revealed sensitivity to silencing of TNK2 and the SRC family kinase, FGR (FIG. 1C), both of which are potently inhibited by dasatinib. We also performed drug sensitivity profiling of two cases with the CSF3R T618I mutation (one CNL, one ETP-T-ALL). In contrast to the truncation mutant drug sensitivity pattern, both cases exhibited sensitivity to inhibitors that target JAK family kinases (including ruxolitinib; Jakafi™, Incyte) but resistance to dasatinib (FIG. 1D, FIG. 6D, 6E). Taken together, the functional genomic data on these three patient specimens suggested two different classes of CSF3R mutations that result in dysregulation of SRC family/TNK2 in the case of truncation mutations or JAK family kinases in the case of membrane proximal mutations. The data also suggest that truncation mutations confer sensitivity to dasatinib but not JAK kinase inhibitors, while the reverse is true for membrane proximal mutant cells.

### Example 4

### Distinct Signaling Pathway Dysregulation Downstream of CSF3R Truncation or Membrane Proximal Mutations

To test the relative transforming capacity of truncation versus membrane proximal mutant CSF3R, a cytokine independent growth assay was performed using the IL3-dependent Ba/F3 cell line (FIG. 1E). Both classes of CSF3R mutations were capable of transforming Ba/F3 cells to IL-3-independent growth, and the membrane proximal mutations (T615A and T618I) transformed cells in this assay substantially faster than the truncation mutants (Q741X and S783fs).

Once the transformation capacity of the CSF3R mutations was confirmed, the differential signaling and drug sensitivity suggested by our functional screening of CSF3R mutant leukemia specimens was investigated. Ba/F3 cells expressing the T618I or S783fs mutations before or after IL-3-independent transformation along with cells expressing WT CSF3R or parental control cells were starved of the IL-3 growth factor and cell lysates were analyzed by immunoblot. The S783fs mutation exhibited higher expression of CSF3R than WT (FIG. 1F), and this difference was magnified after long-term culture in the absence of IL-3, consistent with prior studies showing disruption of receptor endocytosis in the context of truncation mutations (Hunter M G, Blood 93, 440-446 (1999); incorporated by reference herein). After IL-3 withdrawal, CSF3R mutant Ba/F3 cells expressed high levels of endogenous TNK2 and increased phosphorylation of SRC family kinases, providing validation of the initial TNK2 and FGR siRNA sensitivities observed in a patient specimen with CSF3R truncation mutation (FIG. 1C), and suggesting that TNK2 is a previously unrecognized downstream mediator of CSF3R signaling.

Immunoblot analysis for JAK/STAT phosphorylation was then performed. The T618I mutant induced high levels of STAT3 and JAK2 phosphorylation, in sharp contrast to the lower levels induced by the S783fs mutant (FIG. 1F). Together these data indicate that the two classes of CSF3R mutants have different transformation potential and downstream signaling consequences.

### Example 5

### CSF3R Mutant Leukemia can be Targeted with Tyrosine Kinase Inhibitors

To further test the sensitivities of CSF3R truncation and membrane proximal mutants to SRC family kinase/TNK2 or JAK inhibition, mouse bone marrow cells were transduced with CSF3R S783fs, CSF3R T618I or an empty vector control and plated in a colony formation assay. The empty vector control cells, which express endogenous levels of wild-type CSF3R, required 10 nanograms per milliliter (ng/mL) of exogenous GCSF to form colonies; in contrast, CSF3R S783fs mutant cells required 0.4 ng/mL of GCSF to elicit colony formation, and the T618I mutant grew in the absence of any added GCSF. Treatment with dasatinib had a dramatic effect on colony formation in the context of S783fs mutant cells with an IC₅₀ of about 1 nanomolar (nM) (FIG. 2A). Consistent with the results from primary patient samples, the T618I mutant was relatively insensitive to dasatinib (IC₅₀ about 100 nM), and the empty vector control cells were completely insensitive to dasatinib. Interestingly, all cells exhibited similar sensitivity to the JAK kinase inhibitor ruxolitinib with IC₅₀s of about 100 nM (an equivalent sensitivity as cells with defined JAK-dependency) (FIG. 2B) (Quintas Cardama A et al, Blood 114, 3109-3117 (2010); incorporated by reference herein). The ruxolitinib sensitivity of the empty vector and S783fs mutant cells must be understood in the context of exogenous GCSF requirement to stimulate colony growth, where this exogenous GCSF preferentially stimulates JAK/STAT signaling. Indeed, primary patient cells with CSF3R truncation mutation exhibited sensitivity to dasatinib, but no sensitivity to JAK kinase inhibition when cultured in the absence of exogenous GCSF (FIG. 1B, FIG. 1C). In contrast, colony formation of the T618I cells exhibited ruxolitinib sensitivity despite no requirement for exogenous GCSF for colony outgrowth, consistent with sensitivity to JAK kinase inhibition of primary patient cells with CSF3R membrane proximal mutations (FIG. 1D). Taken together, these data demonstrate that CSF3R truncation mutants studied in vitro are sensitive to SRC family/TNK2 inhibitors and membrane proximal mutants are sensitive to JAK kinase inhibitors.

### Example 6

### Clinical Efficacy of Ruxolitinib in a CNL Patient with CSF3R T618I

Primary cells from a CNL patient with CSF3R T618I (Patient #9; Table S3; FIG. S3C) exhibited in vitro hypersensitivity to ruxolitinib (IC5₀: 127 nM; FIG. S3E). Treatment of this patient with ruxolitinib (10 milligrams (mg) taken orally twice daily) resulted in a marked decrease in total white blood cells (WBC) and absolute neutrophil count (ANC) (FIG. 2C). Increasing the dose of ruxolitinib to 15 mg twice daily led to a further decrease in both the WBC and ANC. This treatment also resulted in normalization of the platelet count (FIG. 2D).

TABLE 2
Mutations in CSF3R enriched in patients with neutrophilic
leukemia - leukemia patient samples were sequenced for
CSF3R exons 14-17 encompassing the membrane proximal
extracellular domain, transmembrane, and cytoplasmic domains.
Compound mutations listed in predicted temporal order of
acquisition by leukemic cells.

| | | CSF3R |
|---|---|---|
| Patient # | Diagnosis | Mutation |
| 1 | aCML favored over CNL¹ | D771fs |
| 2 | aCML | G683R² |
| 3 | CNL | S783fs/T615A |
| 4 | aCML | T615A/Y752X |
| 5 | CNL | T618I/D771fs |
| 6 | CNL | T618I/E808K³ |
| 7 | aCML favored over CNL¹ | T618I/W791X |
| 8 | aCML | T618I |
| 9 | CNL | T618I |
| 10 | CNL | T618I |
| 11 | aCML favored over CNL¹ | T618I |
| 12 | CNL | T618I |
| 13 | aCML | T618I |
| 14 | CNL | T618I |
| 15 | CNL | T618I |
| 16 | aCML | T618I |
| 17 | aCML | None |
| 18 | aCML | None |
| 19 | aCML | None |
| 20 | aCML | None |
| 21 | CNL | None |
| 22 | aCML | None |
| 23 | aCML | None |
| 24 | aCML | None |
| 25 | aCML | None |
| 26 | aCML | None |
| 27 | aCML | None |
| 28 | aCML | None |

¹aCML favored over CNL because of the presence of granulocytic dysplasia and/or prior WBC differential showing >10% immature precursors.
²G683R not evaluated in Ba/F3 cells.

TABLE 3
Highest recorded WBC reported in/μL. WBC reported in/μL. ANC reported
in/μL. Hb reported in g/dL. Platelets reported in/μL. MCV reported in fL.

| | | % Seg + Band | | | | | | |
|---|---|---|---|---|---|---|---|---|
| ID | Gender | Neutrophils/% | Highest | | | | | |
| # | Age | Precursors | WBC | WBC | ANC | Hb/Hct | Platelets | MCV |
| 1 | M/67 | 82(82 + 0)/<5 | 20,000 | 15,900 | 13,038 | 9.5/28.2 | 210,000 | 100.9 |
| 2 | F/50 | 60(58 + 12)/10 | 42,000 | 13,200 | 7700 | 14.1/42.6 | 54,000 | 89.5 |
| 3 | M/73 | 82(82 + 0)/<5 | 64,000 | 8100 | 6642 | 9.2/26.4 | 415,000 | 97.7 |
| 4 | M/70 | 42(20 + 22)/48 | 270,000 | 37,100 | 15,582 | 9.0/27.2 | 126,000 | 95.9 |
| 5 | F/76 | 77(47 + 30)/19 | 94,500 | 94,500 | 72,750 | 12.3/35.0 | 288,000 | 91.3 |
| 6 | M/73 | 94(94 + 0)/0 | 271,000 | 8300 | 7802 | 10.3/30 | 30,000 | 100.9 |
| 7 | M/71 | 94(89 + 5)/0 | 83,800 | 56,700 | 53,298 | 10.4/32.3 | 215,000 | 90.5 |
| 8 | M/88 | 81(65 + 16)/7 | 175,900 | 38,100 | 24,800 | 8.8/24.5 | 98,000 | 92.3 |
| 9 | M/53 | 86(81 + 5)/9 | 108,100 | 96,500 | 87,800 | 10.8/33.6 | 65,000 | 94.9 |
| 10 | F/73 | 96(96 + 0)/0 | 178,600 | 178,600 | 171,456 | 7.5/22.2 | 83,000 | 111.3 |
| 11 | M/77 | 95(75 + 20)/<5 | 39,100 | 39,100 | 24,992 | 8.4/26.0 | 69,000 | 108.7 |
| 12 | M/69 | 87(80 + 7)/7 | 250,000 | 115,500 | 100,485 | 8.3/NA | 129,000 | N/A⁴ |
| 13 | F/73 | 81(58 + 23)/6 | 211,000 | 98,700 | 79,947 | 11.2/34.6 | 271,000 | 93.2 |
| 14 | M/57 | 93(93 + 0)/0 | 63,800 | 48,500 | 45,100 | 11.0/32.6 | 108,000 | 92.2 |
| 15 | M/67 | 88(83 + 5)/3 | 272,400 | 163,000 | 154,900 | 8.8/25.8 | 22,000 | 90.5 |
| 16 | F/74 | 72(62 + 10)/24 | 177,000 | 105,000 | 75,600 | 9.9/29.6 | 88,000 | 78.2 |
| 17 | F/48 | 64(40 + 15)/7 | 96,000 | 80,100 | 51,264 | 10.2/32.5 | 204,000 | 96.3 |
| 18 | F/73 | 60(53 + 7)/26 | 252,600 | 133,200 | 79,920 | 8.3/27.8 | 117,000 | 85.1 |
| 19 | F/62 | 64(64 + 0)/0 | 42,900 | 12,900 | 8,256 | 12.1/36.3 | 378,000 | 98.7 |
| 20 | M/87 | 50(36 + 14)/43 | 47,000 | 38,900 | 19,450 | 9.3/27.2 | 50,000 | 91.7 |
| 21 | M/86 | 92(92 + 0)/0 | 130,000 | 73,200 | 67,344 | 11.2/36.7 | 190,000 | 81.4 |
| 22 | M/86 | 48(42 + 6)/49 | 114,000 | 114,000 | 54,720 | 7.7/23.7 | 8,000 | 90.8 |
| 23 | M/80 | 58(30 + 28)/18 | 64,100 | 64,100 | 37,178 | 13.1/38.6 | 180,000 | 86.3 |
| 24 | M/74 | 89(89 + 0)/6-20 | 41,300 | 41,300 | 36,757 | 12.1/38.2 | 38,000 | 104.0 |
| 25 | F/49 | 48(32 + 16)/36 | 69,100 | 69,100 | 33,168 | 9.8/28.2 | 223,000 | 99.4 |
| 26 | M/71 | 51(35 + 26)/20 | 102,000 | 102,000 | 52,020 | 9.1/10.3 | 272,000 | 101 |
| 27 | F/76 | 69(63 + 6)/9 | 65,000 | 18,000 | 12,420 | 9.2/26.1 | 295,000 | 84.3 |

TABLE 4
CSF3R compound mutations are on the same allele

| ID# | CSF3R Mutations | Frequency |
|---|---|---|
| 3 | None | 3/9 |
| 3 | T615A only | 0/9 |
| 3 | S783fs only | 4/9 |
| 3 | S783fs/T615A | 2/9 |
| 5 | None | 2/11 |
| 5 | T618I only | 3/11 |
| 5 | D771fs only | 0/11 |
| 5 | T618I/D771fs | 6/11 |

RNA was isolated from cryopreserved patient samples. cDNA was then synthesized and used to amplify a region of CSF3R encompassing both mutations. The PCR products were TOPO TA cloned (Invitrogen), transformed into E. coli, and individual clones were isolated from bacterial colonies and sequenced. The frequency of the mutations is listed as a fraction of the total colonies sequenced. Both patients had clones with both the membrane proximal point mutations and the truncation mutations, indicating that they were on the same allele. Patient #3 had some clones with only the S783fs mutation but not the T615A mutation, indicating that the S783fs mutation most likely arose first. This is consistent with the sequencing results from genomic DNA indicating that the T615A mutation was present at very low frequency (FIGS. 5A-5C). Patient #5 had clones with T618I only but not D771fs only, indicating that the T618I mutation likely preceded the D771fs mutation.

### Example 7

### Additional Studies

Since the priority date of this application, this disclosure was published 9 May 2013 as Maxson J E et al, N Engl J Med 368, 1781-1790, which is incorporated by reference herein.

Furthermore, a study was performed that showed CSF3R T618I mutation was detected in 83% of patients with CNL (Pardanani A et al, Leukemia 27, 1870-1873 (22 Apr. 2013); incorporated by reference herein.)

Also, a new algorithm for diagnosis and treatment of neutrophilia was developed based in part upon this disclosure in Gotlib J et al, Blood 122, 1707-1711 (Sep. 5, 2013); incorporated by reference herein.

In addition, introduction of a CSF3R T618I mutation into mice results in a neutrophilic neoplasia that is responsive to treatment with JAK inhibitors such as ruxolitinib (Fleischman A G et al, Blood 122, 3628-3531 (21 Nov. 2013); incorporated by reference herein.

Finally, it was shown that the CSF3R T615 and T618 mutations result in rendering CSF3R ligand independent by preventing O-glycosylation of CSF3R resulting in increased receptor dimerization and signaling (Maxson J E et al, J Biol Chem 289, 5280-5287 (28 Feb. 2014); incorporated by reference herein.)

1. A method of treating a chronic neutrophilic leukemia or atypical chronic myeloid leukemia in a subject, the method comprising:
obtaining a sample from the subject, where the sample comprises one or more leukemia cells;
amplifying a nucleic acid fragment comprising SEQ. ID NO: 8 from the sample; and
administering a pharmaceutical composition comprising ruxolitinib to the subject, provided that the amplified nucleic acid fragment comprises a mutation that corresponds to a mutation of T618 in SEQ ID NO: 1, thereby treating the chronic neutrophilic leukemia or atypical chronic myeloid leukemia.

2. The method of claim 1 further comprising performing Sanger sequencing on the nucleic acid fragment.

3. The method of claim 1 further comprising amplifying the nucleic acid fragment using a first oligonucleotide comprising SEQ. ID NO: 2 and a second oligonucleotide comprising SEQ. ID NO: 3 and where the first oligonucleotide and second oligonucleotide are not more than 45 nucleotides in length.

4. The method of claim 1 where the T618 mutation is a T618I mutation.

5. A method of treating a chronic neutrophilic leukemia in a subject, the method comprising:
obtaining a sample from the subject, where the sample comprises one or more leukemia cells;
amplifying a nucleic acid fragment comprising SEQ. ID NO: 9 from the sample; and
administering a pharmaceutical composition comprising dasatinib to the subject, provided that the amplified nucleic acid fragment comprises a mutation that corresponds to a frameshift mutation at S783 of SEQ. ID NO: 1, thereby treating the chronic neutrophilic leukemia or atypical chronic myeloid leukemia in the subject.

6. The method of claim 5 further comprising performing Sanger sequencing on the nucleic acid fragment.

7. The method of claim further comprising amplifying the nucleic acid fragment using a first oligonucleotide comprising SEQ. ID NO: 4 and a second oligonucleotide comprising SEQ. ID NO: 5 and where the first oligonucleotide and second oligonucleotide are not more than 45 nucleotides in length.

8. A method of selecting a subject with chronic neutrophilic leukemia or atypical chronic myeloid leukemia for inclusion in a clinical trial, the method comprising:
obtaining a sample from the subject, the sample comprising leukemia cells;
amplifying a nucleic acid fragment comprising SEQ. ID NO: 8 from the sample; and
including the subject in the clinical trial provided that the amplified nucleic acid fragment comprises a mutation that corresponds to a mutation of T618 in SEQ. ID NO: 1 and provided that the pharmaceutical composition to be tested in the clinical trial comprises a Janus kinase inhibitor.

9. The method of claim 8 further comprising performing Sanger sequencing on the nucleic acid fragment.

10. The method of claim 8 further comprising amplifying the nucleic acid fragment using a first oligonucleotide comprising SEQ. ID NO: 2 and a second oligonucleotide comprising SEQ. ID NO: 3 and where the first oligonucleotide and second oligonucleotide are not more than 45 nucleotides in length.

11. The method of claim 8 where the T618 mutation is a T618I mutation.


SEQUENCE LISTING (9 sequences)

SEQ ID NO: 1 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 2 - DNA, 35 bp, Artificial Sequence
  Exon 14 Forward PCR Primer
gtaaaacgacggccagtccacggaggcagctttac

SEQ ID NO: 3 - DNA, 38 bp, Artificial Sequence
  Exon 14 Reverse PCR Primer
caggaaacagctatgaccaaatcagcatcctttgggtg

SEQ ID NO: 4 - DNA, 37 bp, Artificial Sequence
  Exon 17 Forward PCR Primer
gtaaaacgacggccagtagtggcccaaagacacagtc

SEQ ID NO: 5 - DNA, 38 bp, Artificial Sequence
  Exon 17 Reverse PCR Primer
caggaaacagctatgaccgggagtcccataacagctca

SEQ ID NO: 6 - DNA, 17 bp, Artificial Sequence
  M13 forward primer
gtaaaacgacggccagt

SEQ ID NO: 7 - DNA, 18 bp, Artificial Sequence
  M13 Reverse Primer
caggaaacagctatgacc

SEQ ID NO: 8 - DNA, 141 bp, Homo sapiens
ccgccatcctgaatgcctcctcccgtggctttgtcctccatggcctggagcccgccagtctgtatcacatccacctcatggctgccagccaggctggggccaccaacagtacagtcctcaccctgatgaccttgaccccag

SEQ ID NO: 9 - DNA, 734 bp, Homo sapiens
gatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagggcttcctggggttcccttcttgggcctgcctcttaaaggcctgagctagctggagaagaggggagggtccataagcccatgactaaaaactaccccagcccaggctctcaccatctccagtcaccagcatctccctctcctcccaatctccataggctgggcctcccaggcgatctgcatactttaaggaccagatcatgctccatccagccccacccaatggccttttgtgcttgtttcctataacttcagtattgtaaac